(19) United States
(12) Patent Application Publication
(10) Pub. No.: US 2026/0306825 A1
(43) Pub. Date: Oct. 8, 2026

(54) NON-OBSTRUCTIVE MECHANICAL CIRCULATORY ASSIST DEVICES, SYSTEMS, AND METHODS OF USE

(71) Applicant: Regents of the University of Minnesota, Minneapolis, MN (US)

(72) Inventors: Eric L. Severson, Arden Hills, MN (US); Takahiro Noguchi, Minneapolis, MN (US); Pranava Sinha, Edina, MN (US)

(73) Assignee: Regents of the University of Minnesota, Minneapolis, MN (US)

(21) Appl. No.: 19/640,146

(22) Filed: Apr. 6, 2026

Related U.S. Application Data
(60) Provisional application No. 63/783,403, filed on Apr. 4, 2025.

(51) Int. Cl. A61M 60/35; A61M 60/17; A61M 60/216; A61M 60/459; A61M 60/462; A61M 60/802

(52) U.S. Cl. CPC A61M 60/35; A61M 60/17; A61M 60/216; A61M 60/459; A61M 60/462; A61M 60/802; A61M 2205/103; A61M 2210/125

(57) ABSTRACT

A bearingless circulatory assist device including at least one rotor/stator pair and an axial flow impeller. The rotor is hollow, and the rotor/stator pair is configured such that an outer radial surface of the rotor interacts with magnetic fields associated with the corresponding stator to create a normal magnetic force on the rotor. In some examples, the circulatory assist device is a bearingless motor that is capable of producing controllable radial and tilting magnetic forces and passive axial forces to stably levitate. The axial flow impeller is connected to the rotor of rotor/stator pair (e.g., integrated into the center of the rotor). The circulatory assist device can be scaled for implantation in pediatric patients (e.g., to supplement Fontan circulation), or as a mechanical pediatric or adult ventricle assist device (e.g., RVAD).

### CROSS REFERENCE TO RELATED APPLICATIONS

This application claims priority to U.S. Provisional Application No. 63/783,403, filed Apr. 4, 2025, and entitled “NON-OBSTRUCTIVE MECHANICAL CIRCULATORY ASSIST DEVICES, SYSTEMS, AND METHODS OF USE”, the entire teachings of which are incorporated herein by reference.

### BACKGROUND

The present disclosure relates to mechanical circulatory assist devices. More particularly, it relates to non-obstructive mechanical circulatory assist devices and related methods of use that are scalable, for example, for implantation in pediatric patients and/or for use as a ventricular assist device or a venous assist device.

Mechanical circulatory assist devices or pumps can be used for short and/or long term treatment of various heart-related maladies. For example, congestive heart failure, in which the heart fails to pump blood at a rate commensurate with the requirements of metabolizing tissue, can be treated, in some instances, by a ventricular assist device that operates to supplement heart function. A ventricular assist device (VAD) is a device that helps pump blood from the lower chambers of the heart to the rest of the body. A left ventricular assist device (LVAD) helps pump blood from the lower left heart chamber, called the left ventricle, to the aorta, the main blood vessel that carries blood from the heart to the rest of the body. A right ventricular assist device (RVAD) supports the right ventricle, pumping blood to the lungs. The VAD pump is implanted inside the patient’s chest, connected to the patient’s heart; a driveline extends from the pump to a controller that operates to control and power the VAD. Other technologies that allow wireless control of the VAD and/or power transfer may be used. Blood passes into the pump and then through a graft attached to the aorta in the case of an LVAD or to the pulmonary artery in the case of an RVAD.

Mechanical circulatory assist devices can also be useful in patients with other heart defects. As a point of reference, patients with normal anatomy and heart failure are typically adequately supported with a conventional VAD. Patients with univentricular congenital heart disease and the like, however, can present even more complex treatment obstacles. The physiology and anatomy of the single ventricle circulation presents challenges to mechanical circulatory support, leading to relative poor outcomes in these patients while awaiting heart transplantation. Single ventricle palliation pathway techniques may be employed to address all congenital heart defects that are not amenable to establishment of a normal two-ventricle circulation. This involves a series of surgical diversions to allow all blood returning from the body to directly and passively flow into the low resistance circuit of the lungs, while the solitary ventricle is used to pump blood into the higher resistance body (systemic) circuit. The procedures are done in a stepwise fashion with diversion of blood flow from the upper body via the superior vena cava (SVC) to the lungs initially (Bidirectional Glenn circulation) followed by addition of the blood from the lower body via the inferior vena cava (IVC) to the lungs (Fontan circulation) via the pulmonary artery (PA). With Fontan circulation, named after the surgeon who developed the operation, the univentricular heart is responsible only for supplying oxygenated blood to the body while deoxygenated blood bypasses the heart and reaches the lungs by passive flow directly from the vena cava to the pulmonary arteries. FIG. 1 shows a diagram of a conventional Fontan circulation in a single ventricle (SV) patient after the surgical procedure is complete.

The Fontan operation comprises about 9% of the approximately 10,000 congenital cardiac surgical procedures performed in the US annually. Significant improvements in the outcomes of the Fontan operation and improved long-term survival in these patients have led to many patients surviving to early adulthood. While this is a viable circulation, absence of a ventricle to pump blood to the lungs limits the efficiency of this circulation, and inefficiencies of the Fontan circulation can result in suboptimal exercise capacity, long-term morbidity, and late mortality. Delayed Fontan circulation failure is seen in a high proportion of patients leading to frequent reinterventions or death over 30 years. In addition to overall low cardiac output, these patients also have chronically elevated venous pressure leading to congestive changes in their liver, kidneys and gut, which presents as hepatic fibrosis/cirrhosis and protein losing enteropathy.

It has been suggested that a mechanical circulatory assist device might be used to assist or support cavopulmonary Fontan circulation, for example in the form of a venous assist device as described in PCT Publication No. 2021/138233, the entire teachings of which are incorporated herein by reference. By way of further background, mechanical circulatory assist or support for a Fontan circulation can be broadly grouped into two strategies: subpulmonary support in which a mechanical pump in place of a subpulmonary ventricle, and systemic support in which assistance is provided to the single functional ventricle.

With these and other mechanical circulatory assist devices, technology to provide mechanical circulatory support in pediatric patients remains relatively undeveloped. Existing approaches often rely on decades-old, large pulsatile pumps that need to be placed outside the body and connected to the child’s heart using relatively large tubes. While effective in short term, these patients typically need to remain in the intensive care unit, and experience elevated complication rates with increasing duration of use. Centrifugal pump technology available for use in adults and older patients has limitations and not miniaturized enough yet for use in the smaller pediatric patients. Currently there is no FDA-approved LVAD for smaller children permitting routine hospital discharge. As a result, such patients often remain hospitalized for extended periods, sometimes for months, while awaiting heart transplantation, thereby imposing significant burdens on patients, families and healthcare systems.

Rotary axial pumps that can be miniaturized enough for use in children have been considered, but require mechanical bearings; this requirement, in turn, leads to areas of stasis, turbulence and blood injury accounting for significant complications such as strokes, blood clots and damage to blood components and inflammation. Additional challenges are imposed by the very complex congenital heart defect patients who fall in the category of Fontan circulation. While it has been suggested to utilize a mechanical circulatory assist device to support these patients, presently there are no artificial blood pumps available for this circulation. Axial flow pumps have been devised that could support a failing Fontan circulation that utilize mechanical bearings. However, to improve the safety of the device, the drawbacks of mechanical bearing should be addressed. Moreover, circulatory assist devices or pumps designed for the left heart (LVAD) are typically not suitable for supporting Fontan circulation or right ventricular assistance (e.g., as an RVAD) due to the risks of suck down events leading to pump failure, thromboembolic complications, and abnormal heart rhythm.

### SUMMARY

The inventors of the present disclosure have recognized a need to address one or more of the above-mentioned problems.

Some aspects of the present disclosure are directed to bearingless circulatory assist devices. In some embodiments, the circulatory assist devices of the present disclosure include at least one rotor/stator pair and an axial flow impeller. The rotor of the rotor/stator pair is hollow. Further, the rotor/stator pair is configured such that an outer radial surface of the rotor interacts with magnetic fields associated with the corresponding stator for producing controllable radial and tilting magnetic forces and passive axial forces to stably levitate. The axial flow impeller is connected to the rotor of the rotor/stator pair (e.g., integrated into the center of the rotor). In some examples, the circulatory assist devices of the present disclosure include first and second rotor/stator pairs. In some embodiments, the at least one rotor/stator is provided as part of a bearingless motor having a stator structure that includes windings distributed among stator slots and electrically connected in phases to define a fundamental suspension pole-pair number of 1 (pₛ = 1) and a fundamental motor pole-pair number of at least 4 (p ≥ 4). The circulatory assist devices of the present disclosure are scalable for implantation in pediatric patients and can function as a mechanical ventricular assist device (e.g., RVAD) or as a mechanical venous assist device (e.g., to supplement Fontan circulation). In both instances, the VAD is intended to pump blood to the lungs. By eliminating the need for any mechanical or hydrodynamic bearings, the circulatory assist devices of the present disclosure highly improve the safety concerns associated with conventional VADs and are more hemocompatible, leading to lower risk of clotting, thromboembolic complications and lower trauma to blood and blood components. Further, the circulatory assist devices of the present disclosure are safe and effective for supporting small pediatric patients and those with complex Fontan circulation in some embodiments and/or for adult mechanical support, for example with assistance of the right heart.

### BRIEF DESCRIPTION OF THE DRAWINGS

FIG. 1 is a diagram of conventional Fontan circulation;

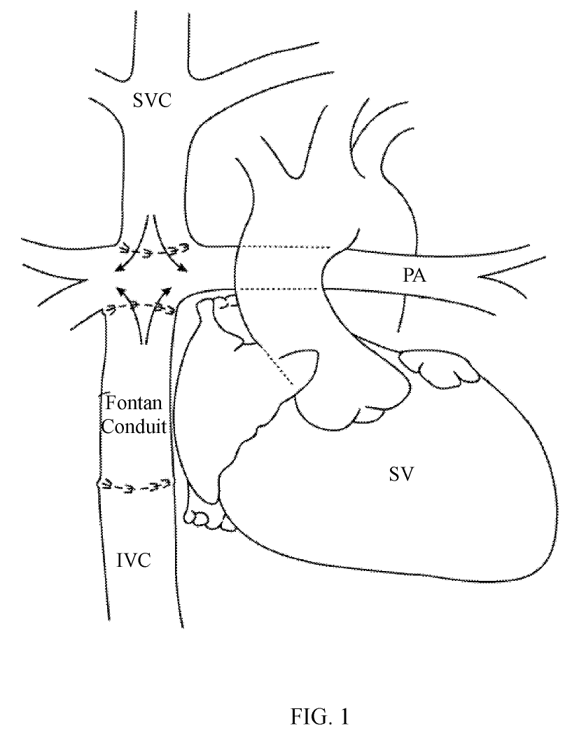

FIG. 2A is a perspective view of a circulatory assist device in accordance with principles of the present disclosure;

FIG. 2B is a cross-sectional view of the circulatory assist device of FIG. 2A;

FIG. 3A is a perspective view of a circulatory assist device in accordance with principles of the present disclosure;

FIG. 3B is a cross-sectional view of the circulatory assist device of FIG. 3A;

FIG. 4A is a perspective cross-sectional view of a circulatory assist device in accordance with principles of the present disclosure;

FIG. 4B is a side cross-sectional view of the circulatory assist device of FIG. 4A;

FIG. 5A is a perspective cross-sectional view of a circulatory assist device in accordance with principles of the present disclosure with portions removed;

FIG. 5B is a perspective cross-sectional view of a circulatory assist device in accordance with principles of the present disclosure with portions removed;

FIG. 5C is a perspective cross-sectional view of a circulatory assist device in accordance with principles of the present disclosure with portions removed;

FIG. 5D is a perspective cross-sectional view of a circulatory assist device in accordance with principles of the present disclosure with portions removed;

FIG. 5E is a perspective cross-sectional view of a circulatory assist device in accordance with principles of the present disclosure with portions removed; and

FIG. 6 is a control circuit for a circulatory assist device in accordance with principles of the present disclosure.

### DETAILED DESCRIPTION

Some aspects of the present disclosure are directed to mechanical circulatory assist devices and methods of use. In some embodiments, the devices of the present disclosure are bearingless pumps, and provide a non-obstructed flow path in the event of rotor failure. As a point of reference, a bearingless pump or motor is defined as an electric machine that simultaneously produces magnetic shear and radial pressure on the same rotor surfaces, usually to magnetically levitate the rotor. There are “bearingless” versions of all types of motors (e.g., permanent magnet, induction, synchronous reluctance, switched reluctance, etc.). In some examples, the mechanical circulatory assist devices of the present disclosure include a hollow rotor with an axial flow impeller integrated into a center of the rotor where the rotor also functions as an electric motor that is capable of producing controllable radial and tilting magnetic forces and passive axial forces to stably levitate. The circulatory assist devices of the present disclosure can be configured and sized to function as a mechanical ventricle assist device or as a mechanical venous assist device (e.g., to support Fontan circulation as a direct replacement of the inferior vena cava or the superior vena cava in the Fontan circulation) and is scalable for implantation in pediatric patients. The bearingless pumps or mechanical circulatory assist devices of the present disclosure can optionally be formatted as, or can include, a bearingless homopolar motor (BHPM), a bearingless multi-monopole motor (BMMM), a bearingless consequent-pole motor (BCPM), or a hybrid version of these configurations designated in the art as a bearingless hybrid consequent-pole motor BHCPM.

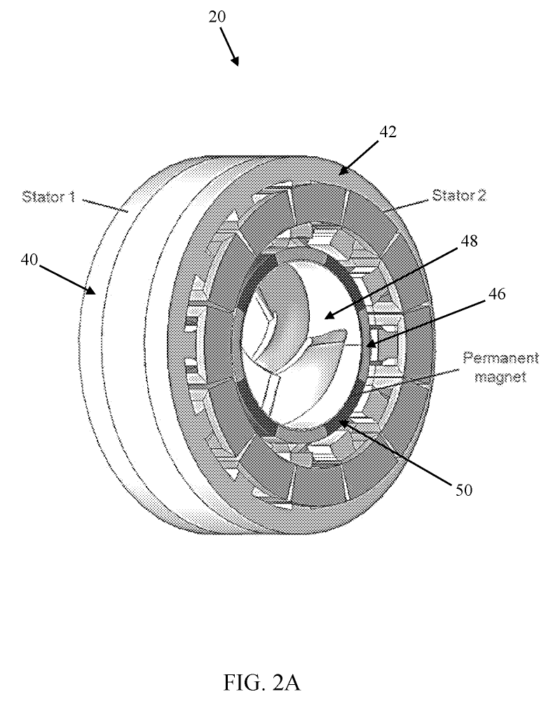

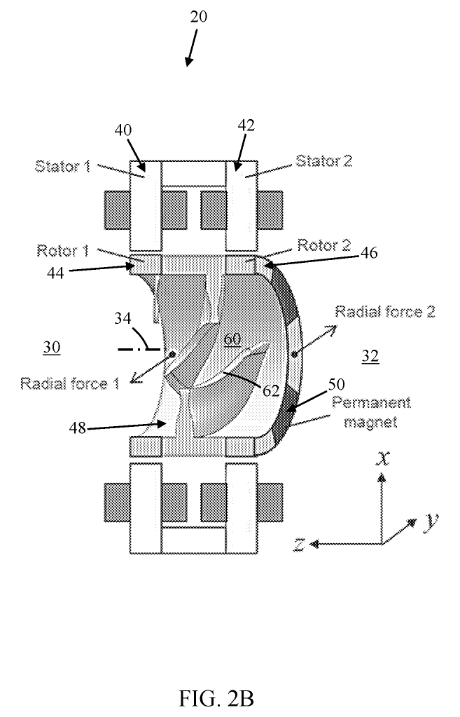

One embodiment of a mechanical circulatory assist device 20 in accordance with principles of the present disclosure is shown in FIGS. 2A and 2B. The circulatory assist device 20 defines an inlet side 30 opposite an outlet side 32 (referenced generally in FIG. 2B), and a central longitudinal axis 34. The circulatory assist device 20 further includes first and second stators 40, 42, first and second rotors 44, 46, and an axial flow impeller 48. The stators 40, 42 are separate from one another and each include at least one stator coil. The rotors 44, 46 each have a hollow configuration, with the impeller 48 being driven by rotation of each of the rotors 44, 46 (e.g., the impeller 48 can be integrated into a center of the rotors 44, 46). The first stator 40 is paired with the first rotor 44, and the second stator 42 is paired with the second rotor 46. Each rotor/stator pair 40/44, 42/46 is magnetically decoupled from the other and is capable of creating an independent x – y force vector. These two independent vectors can be coordinated to control tilting forces in addition to radial forces. Each of the rotors 44, 46 can have surface mounted magnets 50 as shown, or can have a salient pole structure (e.g., synchronous or switched reluctance rotor) or a can be a cage-type rotor. Regardless, the circulatory assist device 20 is a bearingless motor, with an outer radial surface of the rotors 44, 46 interacting with magnetic fields of the corresponding stator 40, 42 to create magnetic force and torque. The torque is used to create rotation on the rotor 44, 46 to cause the pump to operate. The force is controlled to magnetically suspend the corresponding rotor 44, 46. The fields for radial force and torque are both controlled by a stator winding of the corresponding stator 40, 42.

The impeller 48 at least partially defines a central lumen 60 (referenced generally) of the circulatory assist device 20 that extends between the inlet and outlet sides 30, 32 along the longitudinal axis 34. For example, impeller blades 62 each terminate at an end that is radially spaced from the longitudinal axis 34 in at least partially defining the central lumen 60. Stated otherwise, the internally-disposed impeller 48 can be viewed as a hollow shaft. With this construction, under circumstances where the impeller 48 is not rotating (e.g., the rotors 44, 46 cease to rotate), fluid flow between and through inlet and outlet sides 30, 32 can occur via the central lumen 60. Thus, the central lumen 60 is open enough to facilitate fluid passage even if the blades 62 are stopped, minimizing the static pressure drop through the circulatory assist device 20.

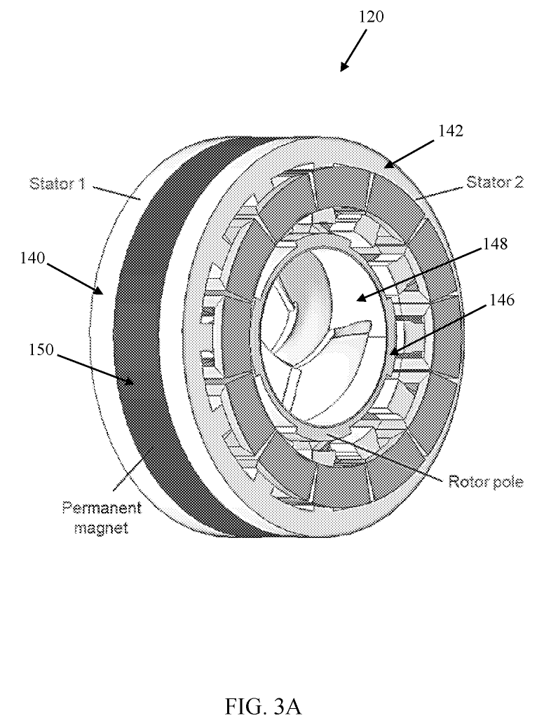

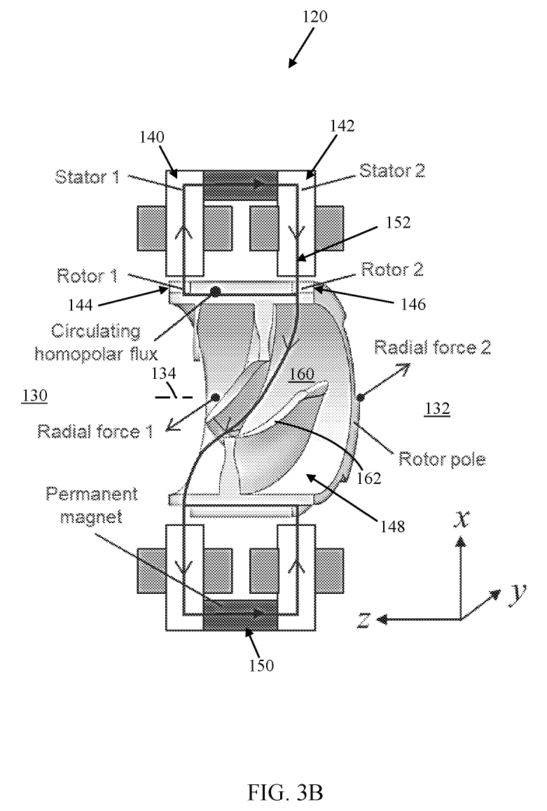

Another embodiment of a mechanical circulatory assist device 120 in accordance with principles of the present disclosure is shown in FIGS. 3A and 3B. The circulatory assist device 120 defines an inlet side 130 opposite an outlet side 132 (referenced generally in FIG. 3B), and a central longitudinal axis 134. The circulatory assist device 120 further includes first and second stators 140, 142, first and second rotors 144, 146, an axial flow impeller 148, and a permanent magnet 150. The stators 140, 142 are separate from one another and each include at least one stator coil. The rotors 144, 146 each have a hollow configuration, with the impeller 148 being driven by rotation of each of the rotors 144, 146 (e.g., the impeller 148 can be integrated into a center of the rotors 144, 146). The first stator 140 is paired with the first rotor 144, and the second stator 142 is paired with the second rotor 146. Each rotor/stator pair 140/144, 142/146 is capable of creating an independent radial force vector that controls net x – y radial forces and tilt. The rotor/stator pairs 140/144, 142/146 are coupled by a circulating homopolar flux (represented by lines 152). In some examples, the homopolar flux can be designed to use a metal of the axial flow impeller 148 to complete its magnetic circuit. The circulatory assist device 120 is a bearingless motor, with an outer radial surface of the rotors 144, 146 interacting with magnetic fields of the corresponding stator 140, 142 to create magnetic force and torque. The torque is used to create rotation on the rotor 144, 146 to cause the pump to operate. The force is controlled to magnetically suspend the corresponding rotor 144, 146. The fields for radial force and torque are both controlled by a stator winding of the corresponding stator 140, 142.

The impeller 148 at least partially defines a central lumen 160 (referenced generally) of the circulatory assist device 120 that extends between the inlet and outlet sides 130, 132 along the longitudinal axis 134. For example, impeller blades 162 each terminate at an end that is radially spaced from the longitudinal axis 134 in at least partially defining the central lumen 160. Stated otherwise, the internally-disposed impeller 148 can be viewed as a hollow shaft. With this construction, under circumstances where the impeller 138 is not rotating (e.g., the rotors 144, 146 cease to rotate), fluid flow between and through inlet and outlet sides 130, 132 can occur via the central lumen 160. Thus, the central lumen 160 is open enough to facilitate fluid passage even if the blades 162 are stopped, minimizing the static pressure drop through the circulatory assist device 120.

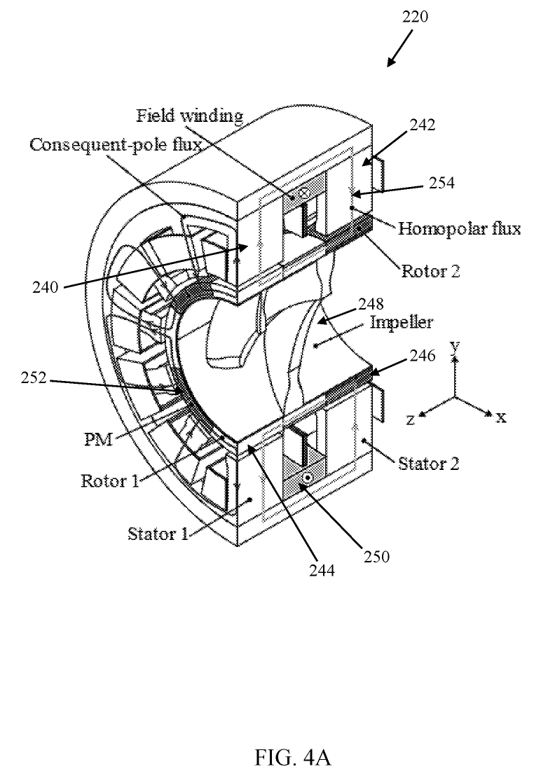

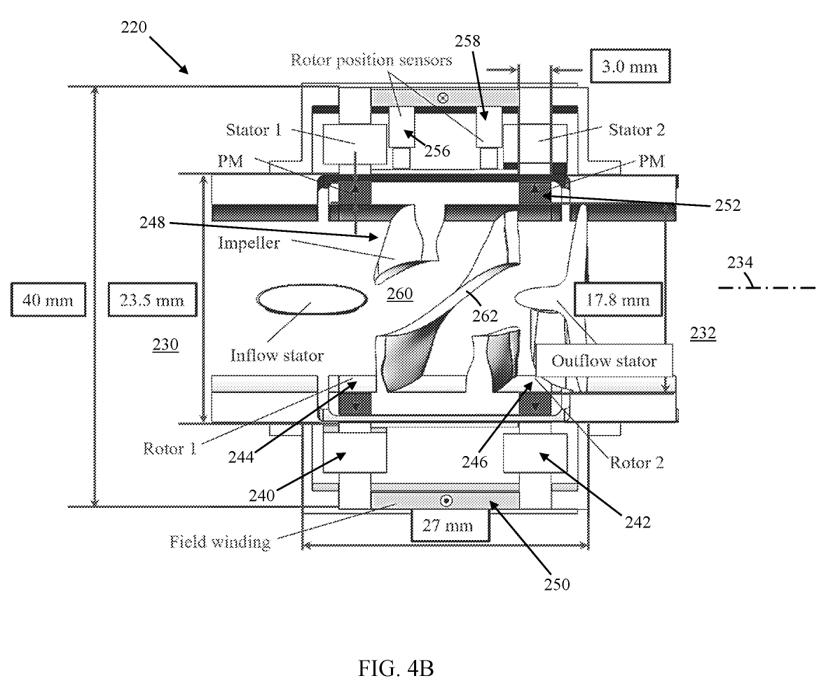

Another embodiment of a mechanical circulatory assist device 220 in accordance with principles of the present disclosure is shown in FIGS. 4A and 4B. As a point of reference, while several dimensions are identified in the view of FIG. 4B, these dimensions are merely examples and are in no way limiting. The circulatory assist device 220 defines an inlet side 230 opposite an outlet side 232 (referenced generally in FIG. 4B), and a central longitudinal axis 234. The circulatory assist device 220 further includes first and second stators 240, 242, first and second rotors 244, 246, an axial flow impeller 248, and a field winding 250. The stators 240, 242 are separate from one another and each include at least one stator coil. The rotors 244, 246 each have a hollow configuration, with the impeller 248 being driven by rotation of each of the rotors 244, 246 (e.g., the impeller 248 can be integrated into a center of the rotors 244, 246). The first stator 240 is paired with the first rotor 244, and the second stator 242 is paired with the second rotor 246. The field winding 250 (or a ring-shaped permanent magnet) is integrated between the first and second first and second stators 240, 242. The first and second rotors 240, 242 contain iron teeth with permanent magnets 252 located in slots between the teeth. Each rotor/stator pair 240/244, 242/246 is capable of creating an independent radial force vector that controls net x – y radial forces and tilt. Thus, the rotor/stator pairs 240/244, 242/246 allows the design to control both radial and tilting movements, while axial displacement is passively stabilized by the rotor magnets 252.

The rotor/stator pairs 240/244, 242/246 are coupled by a homopolar magnetic field flux (represented by lines 254 in FIG. 4A). In some embodiments, the homopolar flux is designed 1) as a superposition of field from the rotor magnets 252 and the field winding 250 to reduce the thickness of the material required to carry an axial field, and 2) to use metal of the axial flow impeller 248 to complete its magnetic circuit.

The circulatory assist device 220 is a bearingless motor (e.g., 3D magnetic suspension), with an outer radial surface of the rotors 244, 246 interacting with magnetic fields of the corresponding stator 240, 242 to create magnetic force and torque. The torque is used to create rotation on the rotor 244, 246 to cause the pump to operate. The force is controlled to magnetically suspend the corresponding rotor 244, 246. The fields for radial force and torque are both controlled by a stator winding of the corresponding stator 240, 242. In some embodiments, first and second rotor position sensors 256, 258 can be provided and are position between the first and second stators 244, 246 to detect a position of the impeller 238 for levitation control. With these and related embodiments, rotational information is not necessary for levitation control. Moreover, at least some of the circulatory assist devices of the present disclosure, such as the circulatory assist device 220, do not include or require an encoder; this attribute, in turn, allows for miniaturization of the device 220 to meet size constraints and makes the design more robust.

The impeller 248 at least partially defines a central lumen 260 (referenced generally) of the circulatory assist device 220 that extends between the inlet and outlet sides 230, 232 along the longitudinal axis 234. For example, impeller blades 262 each terminate at an end that is radially spaced from the longitudinal axis 234 in at least partially defining the central lumen 260. Stated otherwise, the internally-disposed impeller 248 can be viewed as a hollow shaft. With this construction, under circumstances where the impeller 248 is not rotating (e.g., the rotors 244, 246 cease to rotate), fluid flow between and through inlet and outlet sides 230, 232 can occur via the central lumen 260. Thus, the central lumen 260 is open enough to facilitate fluid passage even if the blades 262 are stopped, minimizing the static pressure drop through the circulatory assist device 220. In some embodiments, the circulatory assist device 220 can be configured to provide a large diameter, straight flow through path (i.e., the central lumen 260) of very low pressure drop. This results in low resistance to continue flow should some event result in the impeller 248 not rotating. For example, the circulatory assist device 220 can have a static pressure drop of < 3mm Hg at physiologic flow rates. The hollow construction of the impeller 248 permits some recirculation but beneficially reduces the static pressure drop. The second rotor 246 (on the outlet side 232) contributes to converting velocity to pressure; the first rotor 244 (on the inlet side 230) smooths flow into the impeller 248.

Other bearingless motor formats can be useful as or with the circulatory assist devices of the present disclosure. As a point of reference, the number of rotor pole-pairs can be defined as p and the number of the suspension pole-pairs can be denoted as pₛ. While the most studied configurations are bearingless pₛ = p ± 1 topology, pₛ= 1 machine topologies have also gained attention due to their advantage of not requiring angular position detection for suspension control. The pₛ = 1 machines include: the bearingless homopolar motor (BHMP), the bearingless multi-monopole motor (BMMM), the bearingless consequent-pole motor (BCPM), and the hybrid version of these configurations (BHCPM). In some non-limiting examples, a miniature pₛ= 1 bearingless motor designed for pediatric pump applications which require a compact motor size to accommodate smaller anatomical structures is provided. In some non-limiting examples, a stator of the bearingless motor of the circulatory assist devices of the present disclosure includes windings distributed among stator slots and electrically connected in phases to define a fundamental suspension pole-pair number of 1 (pₛ = 1) and a fundamental motor pole-pair number of at least 4 (p ≥ 4). Optionally, an evaluation algorithm can be employed to rapidly and accurately optimize the twin pₛ = 1 machines, including BHPM, BCPM, and BHCPM, designed for a right ventricular assist device (RVAD) and Fontan circulation assist devices.

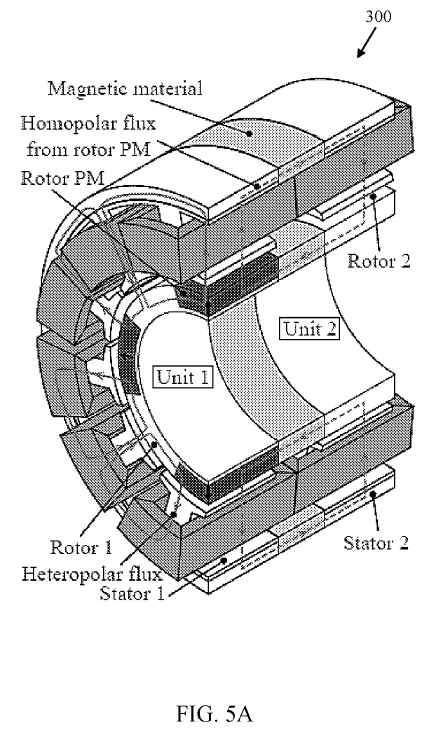

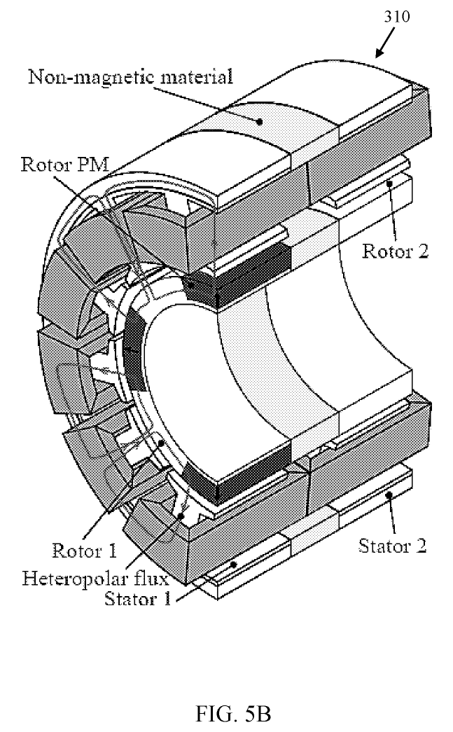

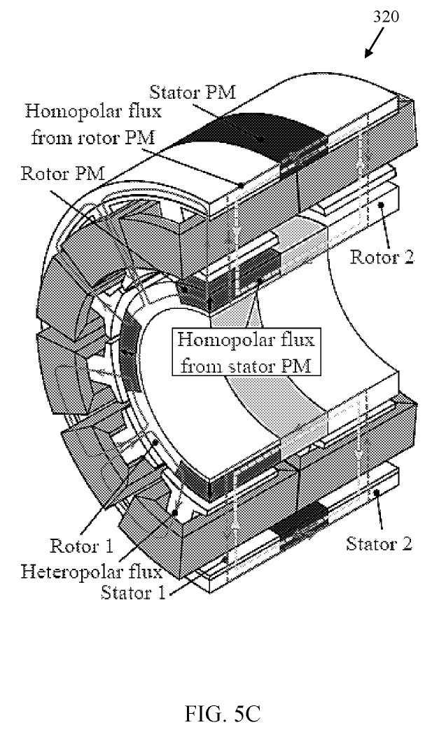

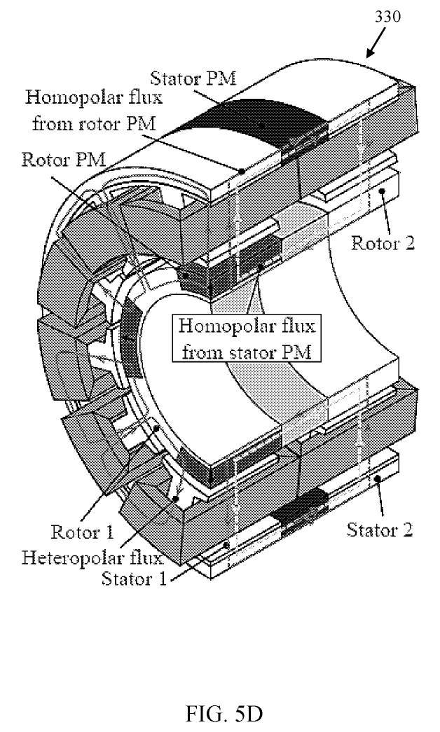

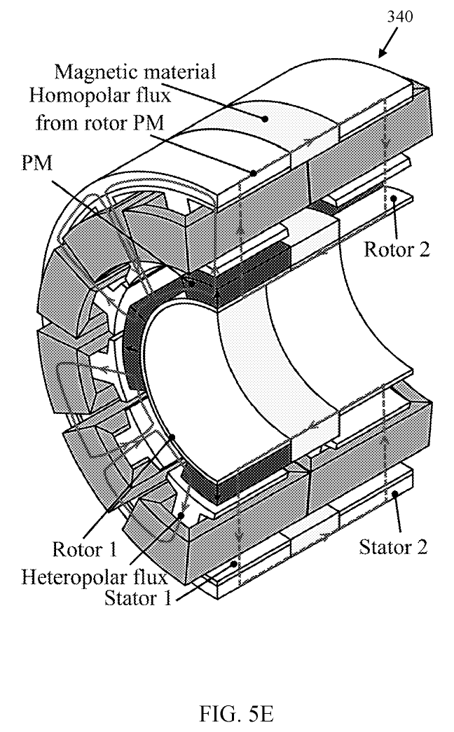

For example, FIGS. 5A-5E are cross-sectional views of portions of other circulatory assist devices 300, 310, 320, 330, 340, respectively, in accordance with principles of the present disclosure and each formatted as a pₛ =1 bearingless machines and do not require an encoder to detect angular position for levitation control. Each of the devices 300, 310, 320, 330, 340 has a twin machine construction defined by two motor units, Unit 1 (with Rotor 1 and Stator 1) and Unit 2 (with Rotor 2 and Stator 2). The motor units, Unit 1 and Unit 2, are connected in tandem to allow active control of radial displacement and tilting. The devices 300, 310, 320, 330, 340 have the same rotor configuration: four pole-pairs, where Unit 1 uses magnets that produce field radially outward and Unit 2 uses magnets with field radially inward. The devices 300, 310, 320, 330, 340 each include an impeller structure that is omitted from the views of FIGS. 5A-5E for ease of illustration, it being understood that an impeller structure akin to the impellers described above is integrated into a center of the Rotors 1, 2.

With the above in mind, the circulatory assist device 300 of FIG. 5A has a BCPM configuration, with permanent magnets (PMs), for example neodymium iron boron (NdFeB) magnets having a grade of approximately N48, embedded with the rotor and magnetized in the same direction to create heteropolar fluxes (represented by solid lines in the view). The material between the stator units is magnetic (e.g., S45C steel), allowing the rotor PMs to create a homopolar flux (represented by dashed lines in the view). With these and related embodiments, some BCPM configurations of the present disclosure include a rotor having a consequent-pole permanent magnet structure where the permanent magnets of the same polarity are positioned between at least four magnetic rotor teeth that are equally distributed circumferentially. In this regard, all rotor magnets within a single rotor unit are facing the same direction (i.e., all radially outward magnets or all radially inward magnets).

The circulatory assist device 310 of FIG. 5B has a BCPM configuration similar to the device 300 of FIG. 5A, except that a non-magnetic material (e.g., SUS304 stainless steel) is used between the two stators. Only heteropolar flux (represented by solid lines in the view) can exist since the non-magnetic material blocks homopolar flux traveling between the units. Each unit behaves as an independent pure BCPM.

The circulatory assist device 320 of FIG. 5C has a BHCPM configuration, with a stator ring permanent magnet (PM), for example neodymium iron boron (NdFeB) magnet having a grade of approximately N48, placed between the two stator units. The rotor PM’s produce heteropolar fluxes (represented by solid lines in the view). The stator PM creates a homopolar flux (represented by dashed lines, one of which is labeled at a top portion of the view) that opposes the homopolar flux (represented by dashed lines, one of which is labeled at a top portion of the view) by the rotor PMs. With these and related embodiments, some BHCPM configurations of the present disclosure include both a consequent-pole rotor structure (magnets between alternating teeth) and a homopolar flux source in the stator.

The circulatory assist device 330 of FIG. 5D has a BHCPM configuration similar to the device 320 of FIG. 5C, except that the polarity of the stator PM is reversed so that the homopolar flux (represented by dashed lines, one of which is labeled at a top portion of the view) it creates is in the same direction as the homopolar flux (represented by dashed lines, one of which is labeled at a top portion of the view) created by the rotor PMs. The rotor PM’s produce heteropolar fluxes (represented by solid lines in the view).

The circulatory assist device 340 of FIG. 5E has a BMMM configuration, with the rotor having a plurality of at least four permanent magnets (PM) of the same polarity separated by non-magnetic gaps, without intervening magnetic rotor teeth. The rotor PM’s produce both heteropolar fluxes (represented by solid lines in the view) and homopolar fluxes (represented by dashed lines in the view). With these and related embodiments, some BMMM configurations of the present disclosure include a rotor having a plurality of at least four permanent magnets of the same polarity separated by non-magnetic gaps, without intervening magnetic rotor teeth.

In other embodiments, the circulatory assist devices of the present disclosure can include or comprise a BHPM configuration, with the stator including a homopolar flux source configured to generate a homopolar bias flux. The BHPM constructions of the present disclosure can optionally include a homopolar flux source having at least one of a permanent magnet or a field coil.

With any of the circulatory assist devices of the present disclosure, such as the devices 20, 120, 220, 300, 310, 320, 330, 340, a diamond-like carbon coating is optionally formed on or applied to blood contacting surfaces. With any of the circulatory assist devices of the present disclosure, such as the devices 20, 120, 220, 300, 310, 320, 330, 340, the impeller can optionally have a variable configuration, including a variable impeller leading edge tip angle that varies from inner diameter to outer diameter, better matching the local tip velocity triangles to expected incoming flow velocity so as to improve flow patterns and reduce blood stresses.

The circulatory assist devices of the present disclosure can be provided as part of a system that further includes a controller programmed to control pump speeds and impeller levitation/suspension. Associated drive electronics, sensors and control logic operate to control the currents that create the necessary forces to magnetically suspend the rotors and torque the rotors to rotate. In some embodiments, the control logic can be configured or programmed to control direct axial flux in each bearingless motor (or stator/rotor pair) to dampen axial vibrations. The circulator assist devices of the present disclosure can incorporate the same stator coils (or “combined windings”) to control both force and torque or can incorporate the used of dedicated force coils and dedicated torque coils (or “separated windings”).

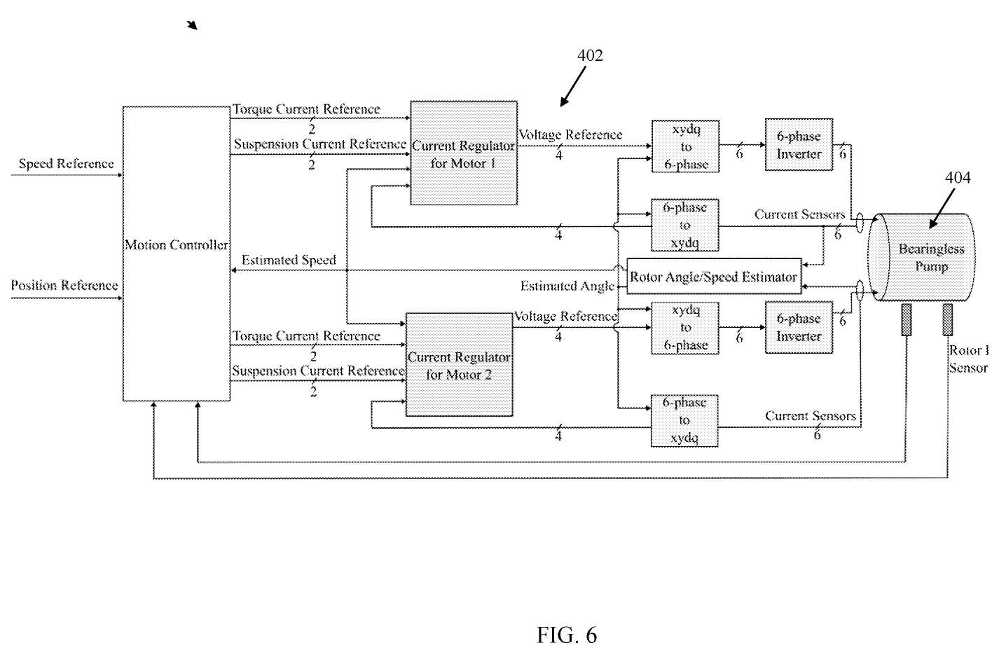

One non-limiting example of portions of a system 400 in accordance with principles of the present disclosure in shown in FIG. 6. The system 400 includes control logic 402 useful with a bearingless circulatory assist device 404 of the present disclosure (e.g., the circulatory assist device 220 (FIG. 4B)). The control logic 402 includes Rotor Position Sensors (e.g., the rotor position sensors 256, 258 (FIG. 4B)) and Current Sensors for the circulatory assist device 404. The control logic 402 is fundamentally composed of an outer loop “Motion Controller” which controls speed and levitation, and an inner loop “Current Regulator” which controls torque and force. The Motion Controller and the Current Regulator can each include or can be commonly defined by a processor coupled to one or more memories. The processor can be a microprocessor, an embedded microprocessor, an embedded controller, a digital signal processor (DSP), etc. The processor is configured to execute program code stored in the memory (e.g., registers, cache, random-access memory, read-only memory, EEPROM, flash memory, USB drives, or the like or combinations thereof). The program code, when executed by the processor, causes the processor to implement the various functions described herein.

For levitation control, measurements from the rotor position sensors are provided to the Motion Controller. These signals are processed by the Motion Controller as shown, which then generates Current References as controller outputs. The speed controller creates a Torque Current Reference, which is the portion of the winding current utilized to make shaft torque, while the levitation control creates the Suspension Current Reference.

In the inner loop current regulation, the feedback signals obtained from the Current Sensors are processed together with the Current References generated by the Motion Controller, producing Voltage References. The Current Sensors measure the winding currents and a Generalized Clarke Transform can be used with in the “6-phase to xydq” block of FIG. 6 to convert these measured currents into the torque and suspension force producing components. The 6-phase inverter then applies the corresponding pulse width modulated (PWM) voltage, allowing current flow through the windings of the bearingless circulatory assist device 402. Since the circulatory assist devices of the present disclosure can consist of two bearingless motor units, current regulation blocks are duplicated, forming two independent control systems. Notably, the rotor angle information required by the Motion Controller can be estimated from the Current Sensors within the inner loop. This function can be performed by the “Rotor Angle/Speed Estimator” block in FIG. 6 to eliminate the need for external sensors such as an encoder or Hall sensors and thereby miniaturize the design.

The circulatory assist devices of the present disclosure can be configured for and implemented as a ventricle assist device (RVAD or LVAD) or as a venous assist device, for example to supplement Fontan circulation. With some examples, the circulatory assist devices of the present disclosure, for example the circulatory assist device 220 of FIGS. 4A and 4B can be designed to assist the cavopulmonary Fontan circulation, serving as a propulsive conduit that can be implanted as a straightforward replacement of the Fontan conduit that connects the IVC to the pulmonary artery. Options to achieve support of both IVC and SVC circulations include a simple extension of the SVC to the IVC proximal the pump inlet. This results in a single pump inlet and outlet and further addresses issues of flow maldistribution with two caval discharges into the pulmonary artery. Alternatively, in patients where routing the SVC would not be feasible, a second pump as an SVC conduit replacement could be used to provide pressure relief/flow augmentation in the SVC circuit and each pump speed could be independently set to suit the circulation it addresses.

The circulatory assist devices, systems and methods of the present disclosure provide a marked improvement over previous designs. The circulatory assist devices of the present disclosure are bearingless and include a hollow rotor with an axial flow impeller integrated into the center of the rotor, where the rotor also functions as part of an electric motor that is capable of producing controllable radial and tilting magnetic forces and passive axial forces to stably levitate. The circulatory assist devices of the present disclosure are scalable for implantation in pediatric patients, and can function as a mechanical ventricle assist device (e.g., RVAD) or as a mechanical venous assist device (e.g., to supplement Fontan circulation). By eliminating the need for any mechanical or hydrodynamic bearings, the circulatory assist devices of the present disclosure highly improve the safety concerns associated with conventional VADs and are more hemocompatible, leading to lower risk of clotting, thromboembolic complications and lower trauma to blood and blood components. Further the circulatory assist devices of the present disclosure are readily scalable to small factors for development of miniaturized axial flow blood pumps that are safe and effective for supporting small pediatric patients and those with complex Fontan circulation in some embodiments. In other embodiments, the circulatory assist devices of the present disclosure are useful for adult mechanical support, for example with assistance of the right heart.

Although the present disclosure has been described with reference to preferred embodiments, workers skilled in the art will recognize that changes can be made in form and detail without departing from the spirit and scope of the present disclosure. For example, while example circulatory assist devices of the present disclosure include two rotor/stator pairs, in other embodiments, circulatory assist devices can include a single rotor/stator pair (e.g., akin to a slice motor that has only a single pair of rotor/stators). Alternatively or in addition, while some examples of the circulatory assist devices of the present disclosure generally reflect a footprint of the rotor of a rotor/stator pair as approximating a footprint of the corresponding stator, in other embodiments, the rotor can extend axially beyond an extent of the stator to enable active control of axial forces.

What is claimed is:

1. A circulatory assist device comprising:
at least one rotor/stator pair;
wherein the rotor of the at least one rotor/stator pair is hollow, and further wherein the at least one rotor/stator pair is configured such that an outer radial surface of the rotor interacts with magnetic fields associated with the corresponding stator to create a normal magnetic force on the rotor; and
an axial flow impeller connected to the rotor of the at least one rotor/stator pair.

2. The circulatory assist device of claim 1, wherein the at least one rotor/stator pair is configured such that interaction with the magnetic fields creates magnetic force and torque.

3. The circulatory assist device of claim 2, wherein the torque creates rotation of the rotor.

4. The circulatory assist device of claim 2, wherein the force is controlled to magnetically suspend the rotor.

5. The circulatory assist device of claim 2, wherein the at least one rotor/stator pair is configured such that fields for radial force and torque are both controlled by a stator winding.

6. The circulatory assist device of claim 1, wherein the device is provided as part of a system that further includes drive electronics, sensors and control logic configured to control currents that create forces to magnetically suspend the rotor of the at least one rotor/stator pair.

7. The circulatory assist device of claim 1, wherein the at least one rotor/stator pair includes a first rotor/stator pair and a second rotor/stator pair.

8. The circulatory assist device of claim 7, wherein the first rotor/stator pair is magnetically decoupled from the second rotor/stator pair.

9. The circulatory assist device of claim 7, wherein the first rotor/stator pair and the second rotor/stator pair are coupled by a circulating homopolar flux.

10. The circulatory assist device of claim 9, wherein a metal material of the axial flow impeller completes the magnetic circuit of the circulating homopolar flux.

11. The circulatory assist device of claim 1, wherein the axial flow impeller defines a continuously open lumen between inlet and outlet sides of the device.

12. The circulatory assist device of claim 1, wherein the circulatory assist device is configured to supplement Fontan circulation.

13. The circulatory assist device of claim 1, wherein the circulatory assist device is configured for use as a right ventricular assist device.

14. The circulatory assist device of claim 1, wherein the stator of the at least one rotor/stator pair comprises windings distributed among stator slots and electrically connected in phases to define a fundamental suspension pole-pair number of 1 (pₛ = 1) and a fundamental motor pole-pair number of at least 4 (p ≥ 4).

15. The circulatory assist device of claim 1, wherein the at least one rotor/stator pair is provided as part of a bearingless consequent-pole motor (BCPM), and further wherein a rotor structure of the BCPM comprises permanent magnets of the same polarity positioned between at least four, circumferentially equidistantly spaced magnetic rotor teeth.

16. The circulatory assist device of claim 1, wherein the at least one rotor/stator pair is provided as part of a bearingless multi-monopole motor (BMMM), and further wherein a rotor structure of the BMMM comprises a plurality of at least four permanent magnets of the same polarity separated by non-magnetic gaps characterized by the absence of intervening magnetic rotor teeth.

17. The circulatory assist device of claim 1, wherein the at least one rotor/stator pair is provided a part of a bearingless homopolar motor (BHPM), and further wherein a stator structure of the BHPM comprises a homopolar flux source configured to generate a homopolar bias flux.

18. The circulatory assist device of claim 17, wherein the homopolar flux source is selected from the group consisting of a permanent magnet and a field coil.

19. The circulatory assist device of claim 1, wherein the at least one rotor/stator pair is provided as part of a bearingless hybrid consequent-pole motor (BHCPM) including a consequent-pole rotor structure and a homopolar flux source in a stator structure of the BHCMP.
